(19) United States
(12) Patent Application Publication
(10) Pub. No.: US 2024/0118276 A1
(43) Pub. Date: Apr. 11, 2024

(54) LATERAL FLOW TEST DEVICE

(71) Applicant: ams-OSRAM AG, Premstaetten (AT)

(72) Inventors: Erik Jan LOUS, Veldhoven (NL); Boon Chong CHEAH, Geldrop (NL); Filip FREDERIX, Molenstede (BE)

(21) Appl. No.: 18/276,885

(22) PCT Filed: Dec. 21, 2021
(86) PCT No.: PCT/EP2021/087016

§ 371 Date: Aug. 11, 2023

(30) Foreign Application Priority Data
Feb. 12, 2021 (GB) 2101978.1

(51) Int. Cl. G01N 33/543

(52) U.S. Cl. CPC G01N 33/54388

(57) ABSTRACT

A lateral flow test device includes a test chamber having a detection aperture. The lateral flow test device also includes an optical detector configured to receive light from an assay test strip through the detection aperture when the assay test strip is provided in the test chamber. The test chamber is configured for manual feed of at least a portion of the assay test strip passed the detection aperture. The optical detector is configured to detect one or more tracking features associated with the assay test strip so as to determine when at least a test line on the assay test strip is detectable through the detection aperture.

### TECHNICAL FIELD OF THE DISCLOSURE

The disclosure relates to a lateral flow test device such as may be useful in biological applications, for example, in the areas of medical, environmental and veterinary diagnostics.

### BACKGROUND OF THE DISCLOSURE

Diagnostic tests are commonly used for identifying diseases. A diagnostic test may be carried out in a central laboratory, whereby a sample, for example blood, is taken from a patient and sent to the central laboratory where the sample is analysed. A different setting for processing samples is at the point where care for the patient is delivered, which is referred to as point-of-care (POC) tests. POC tests allow for a faster diagnosis. Within the POC tests, different technology platforms can be used. A first class of POC tests are high end, microfluidic-based POC tests. These POC tests are mainly used in a professional environment such as hospitals or emergency rooms. A different technology platform is provided by lateral flow test technology. Lateral flow tests are mostly used in the consumer area, such as for pregnancy tests, and are easy to produce and very cost-effective.

Lateral flow tests are very well known as such, but are briefly described by way of background. A lateral flow assay includes a series of capillary beds, such as pieces of porous paper, nitrocellulose membranes, microstructured polymer, or sintered polymer for transporting fluid across a series of pads by capillary forces. A sample pad acts as a sponge and is arranged to receive a sample fluid, and further holds an excess of the sample fluid. After the sample pad is saturated with sample fluid, the sample fluid migrates to a conjugate pad in which the manufacturer has stored the so-called conjugate. The conjugate is a dried format of bio-active particles in a salt-sugar matrix intended to create a chemical reaction between the target molecule (e.g., an antigen) and its chemical partner (e.g. antibody or receptor). While the sample fluid dissolves the salt-sugar matrix, it also mobilizes the bio-active particles and in one combined transport action the sample and conjugate mix with each other while flowing through the capillary beds. The analyte binds to the bio-active particles while migrating further through the third capillary bed. This material has one or more areas, which are called stripes, where a third type of molecule has been immobilized by the manufacturer, in most cases an antibody or receptor addressed against another part of the antigen. By the time the sample-conjugate mix reaches these stripes, analyte has been bound on the bio-active particle and the third type of molecule binds the complex. When more fluid has passed the stripes, particles accumulate on the stripes and the stripes become visible, appear or are generated in a particular colour or with a fluorescent wavelength capability. In this way the stripe is optically detectable by colour or by fluorescent emission detection, respectively.

Typically, there are at least two stripes: a control stripe/line that captures the conjugate and thereby shows that reaction conditions and technology work, and a second stripe, the test stripe/line, that contains a specific capture molecule and only captures those particles onto which an analyte or antigen molecule has been immobilized. This makes the diagnostic result of the test visible for the patient. Some test results rely on the presence of fluorescent particles, which may not be visible to the user but can instead be detected by optical detectors when the stripes are illuminated. After passing the different reaction zones, the fluid enters the final porous material, which is a wick that acts as a waste container.

In summary, lateral flow tests as such are well known and have four key elements: the antibody, the antigen, the conjugate and the complex. Despite these key elements being well established, the terminology used by the skilled person is not always consistent and different terms may refer to the same element. The antibody is also referred to as a receptor, chemical partner, or capture molecule. The antigen is also referred to as an analyte, target molecule, antigen molecule, target analyte or biomarkers. The sample typically contains the analyte, although that is not always the case. The conjugate is also referred to as (analyte) tags, tagging particles, chemical partner, (sample) conjugate mix, bioactive particles or conjugate receptors. Examples of conjugates are fluorescent particles, red particles or dyes. The complex is the combination of the antigen and conjugate. The complex is also referred to as a tagged analyte, or particles onto which the analyte molecule has been immobilised.

In laboratory equipment, either the test strips or optics themselves are configured to move over a pre-defined distance to allow the length of the test strip to be scanned and analysed. However, such equipment is generally large, requires a lot of components and is consequently expensive. A simpler approach uses a mobile phone camera as a detector placed in proximity to a test strip. However, this approach is unreliable as environmental light conditions cause measurement variability and disturbance.

It is therefore an aim of the present disclosure to provide a lateral flow test device that address one or more of the problems above or at least provides a useful alternative.

### SUMMARY

In general, this disclosure proposes to overcome the above problems by providing a simple and cost-effective lateral flow device whereby the test strip is manually fed through the device whilst the location of the test strip is monitored to ensure readings can be taken at the desired positions, for example, for the test line and control line.

Advantageously, this arrangement allows for a small and relatively cheap disposable device, which can still provide reliable test results.

According to a first aspect of the present disclosure, there is provided a lateral flow test device comprising:
a test chamber having a detection aperture; and
an optical detector configured to receive light from an assay test strip through the detection aperture, when the assay test strip is provided in the test chamber;
wherein the test chamber is configured for manual feed of at least a portion of the assay test strip passed the detection aperture; and
wherein the optical detector is configured to detect one or more tracking features associated with the assay test strip so as to determine when at least a test line on the assay test strip is detectable through the detection aperture.

Thus, embodiments of this disclosure provide a lateral flow device in which manual movement of the test strip position can be tracked using the same optical detector as used for detecting results from the test line. As such, a relatively cheap and compact lateral flow device is proposed.

The tracking features may comprise one or more of: a mark, a symbol, a shape, a protuberance, an indentation, a cutout, a colour, a material, a roughness, a thickness, a transparency or another property of the assay test strip. Advantageously, the property used for the tracking of the assay test strip is detectable by the same optical detector as used for detecting light from the test line.

The optical detector may be further configured to determine when a control line on the assay test strip is detectable through the detection aperture. In fact, any number of test lines or control lines may be located and measured on a single test strip.

The test chamber may be configured to prevent or minimize ambient light from reaching the detection aperture. This is important in enabling accurate results without interference from variable ambient light or other environmental conditions.

The test chamber may comprise an entrance aperture for entrance of the assay test strip into the device and an entrance obscurer for preventing or minimizing ambient light from entering the entrance aperture.

The entrance obscurer may comprise one or more of: a curtain, a screen and a brush.

The test chamber may comprise an exit aperture for exit of the assay test strip from the device and an exit obscurer for preventing or minimizing ambient light from entering the exit aperture.

The exit obscurer may comprise one or more of: a curtain, a screen and a brush.

It should be noted that although the two apertures have been described as being one entrance aperture and one exit aperture, in some embodiments, it may not matter which side of the device the assay test strip is inserted from. In other words, the method may allow insertion of the assay test strip via the entrance aperture or the exit aperture. Equally, the method may allow removal of the assay test strip via the entrance aperture or the exit aperture. The names of the apertures therefore denote their purpose at any point in time and may not be limited to that particular purpose during each use of the device.

The lateral flow test device may comprise a single optical emitter configured to emit light onto the assay test strip when provided in the test chamber. The emitter may be used to aid tracking of the movement/position of the test strip and/or to provide light for reflectance or transmission measurements on the test/control line. In embodiments, where no optical emitter is provided, fluorescence of a test material may be detected by the optical detector.

The lateral flow test device may be configured to use digital image correlation to track a position of the assay test strip within the test chamber. For example, a technique similar to that employed in an optical computer mouse may be used to track the position of the test strip relative to the detection aperture. In some embodiments, this may involve capturing successive images and correlating said images to find a maximum correlation between pixel intensity subsets, which relate to a translational shift between the images.

The optical detector may be configured to detect two or more tracking features associated with the assay test strip and the device may be further configured to determine a speed of movement of the assay test strip based on a time of detection of each of the two or more tracking features.

A single optical detector may be provided to detect the one or more tracking features and to receive light from at least the test line of the assay test strip.

The lateral flow test device may be configured as a point-of-care device.

The lateral flow test device may be configured to detect one or more of: coronavirus; coronavirus antibodies. However, a large number of other assays may be employed to suit many different applications.

According to a second aspect of the present disclosure, there is provided a method of operating a lateral flow test device comprising:
manually feeding at least a portion of an assay test strip passed a detection aperture in a test chamber;
detecting, using an optical detector, one or more tracking features associated with the assay test strip;
determining, based on the detection of the one or more tracking features, when at least a test line on the assay test strip is detectable through the detection aperture; and
receiving light, at the optical detector, from the test line of the assay test strip.

In some embodiments, the optical detector may be configured to continuously collect data (e.g. by taking a series of intensity measurements of the received light) as the assay test strip is moved through the device. The collected data may then be correlated with information obtained from detection of the one or more tracking features (which may or may not comprise tracking speed information). In other words, the time when the test line is detectable through the detection aperture can be determined using the tracking features, and the collected data taken at that time can be analysed to obtain a test measurement.

In some embodiments, the lateral flow test device may be configured to detect: 1) a reference point on the assay test strip (e.g. in the form of the assay test strip leading edge; a control line which is always visible; or a mark or other tracking feature as described above) and 2) a tracking speed of the assay test strip movement. If the location of the test line relative to the reference point is known, the tracking speed can be used to determine when the test line would have been passed under the detection aperture and therefore the intensity measurement taken at that time can be determined.

In some embodiments, the tracking speed may not be required. For example, if the tracking feature is aligned with the test line (e.g. such that the test line is detectable through the detection aperture at the same time as the tracking feature), detection of the tracking feature will itself indicate the time of the test measurement.

According to a third aspect of the present disclosure, there is provided an assay test strip for use with the lateral flow test device of the first aspect, wherein the assay test strip comprises at least a test line and one or more tracking features to aid location of the test line such that the test line is detectable through the detection aperture of the device.

Lateral flow test devices according to the present disclosure are advantageously: simple to use, compact, cost-effective and potentially disposable due to minimal required components (e.g. one aperture, one detector and one emitter); able to track position of a manually fed assay test strip using the same detector as used for taking measurements of the test/control lines; able to track and measure reliably from any number of test/control lines; and able to provide accurate results, in reflectance/transmission or fluorescent measure modes due to provision of a dark test chamber which minimises interference from ambient/environmental light conditions.

### BRIEF DESCRIPTION OF THE PREFERRED EMBODIMENTS

Some embodiments of the disclosure will now be described by way of example only and with reference to the accompanying drawings, in which:

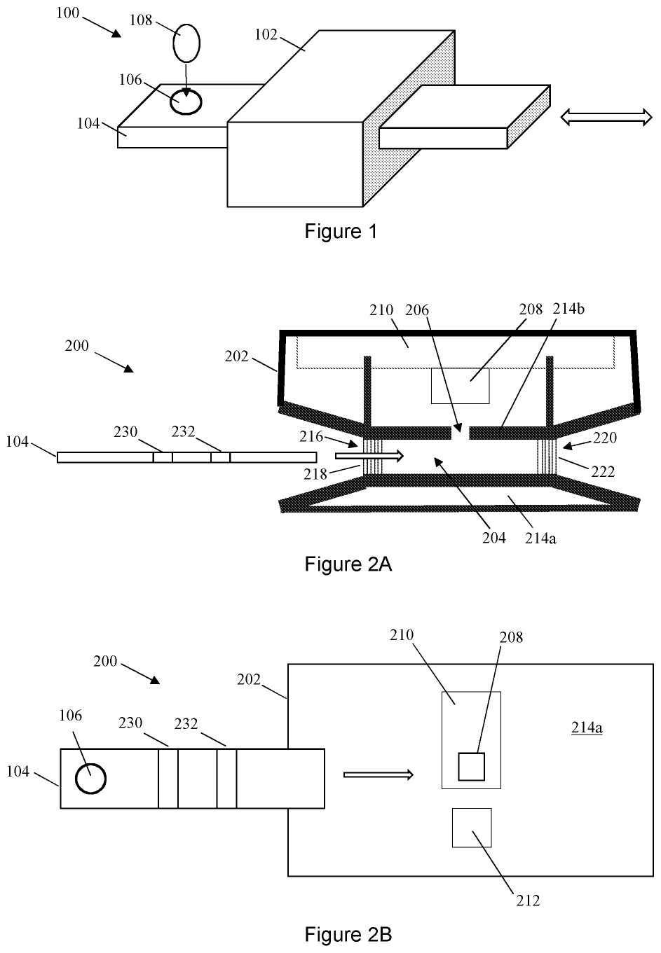

FIG. 1 shows a schematic view of a lateral flow test device with an assay test strip inserted in accordance with the present disclosure;

FIG. 2A shows a side cross-sectional view through a lateral flow test device in accordance with the present disclosure, prior to insertion of the assay test strip;

FIG. 2B shows a schematic plan view and partial section of the set-up of FIG. 2A;

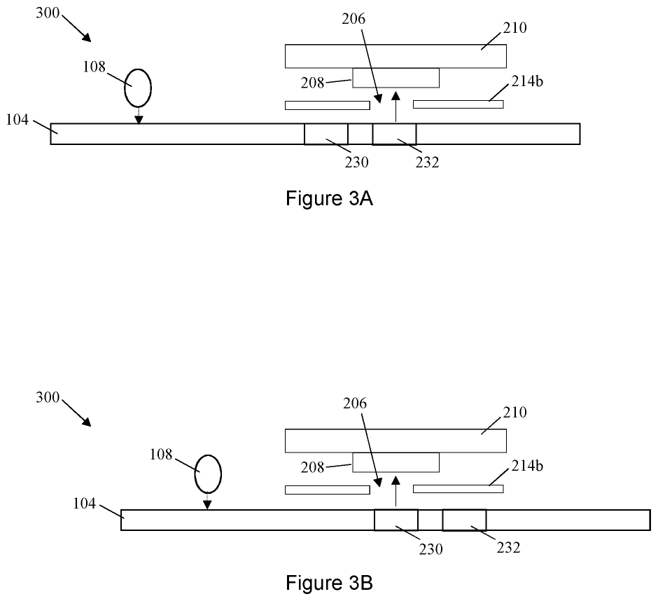

FIG. 3A shows a simplified side view of the set-up if FIG. 2A, with the assay test strip inserted such that the control line is detectable;

FIG. 3B shows a simplified side view of the set-up if FIG. 2A, with the assay test strip inserted such that the test line is detectable; and

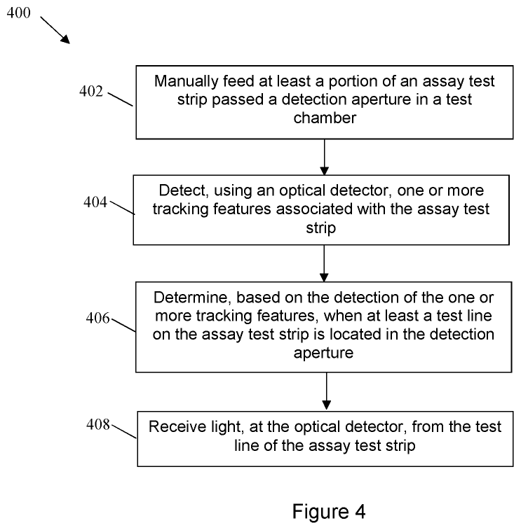

FIG. 4 shows a flow diagram of the steps involved in a method of operating a lateral flow test device in accordance with the present disclosure.

### DETAILED DESCRIPTION OF THE PREFERRED EMBODIMENTS

Generally speaking, the disclosure provides a lateral flow test device and method of operation such that manual insertion of an assay test strip can be monitored to ensure that the detector takes readings from the desired locations on the assay test strip (e.g. the test line and control line).

Some examples of the solution are given in the accompanying figures.

FIG. 1 shows a schematic set-up 100 comprising a lateral flow test device 102 with an assay test strip 104 inserted in accordance with the present disclosure. Much of the detail of the lateral flow test device 102 and assay test strip 104 will be described below. However, as shown in FIG. 1, the assay test strip 104 comprises a sample port 106 onto which a sample 108 is provided in use. The sample 108 may be in the form of a drop of blood, urine or salvia, for example. The sample port 106 comprises a pad of wicking material configured to soak up the sample 108 and encourage flow of the sample along the assay test trip 104 due to capillary forces.

The assay test strip 104 is manually inserted, typically in a horizontal plane, into a slot (not shown) on a first side of the lateral flow test device 102. In the schematic set-up 100, the assay test strip 104 is able to pass completely though the lateral flow test device 102 to exit from a corresponding slot on a second, opposite side of the lateral flow test device 102. Accordingly, the assay test strip 104 may be fed into one side of the lateral flow test device 102 and removed from the opposite side. As shown in FIG. 1, central portion of the assay test strip 104 is within the lateral flow test device 102, while a leading end is visible exiting from the lateral flow test device 102 and a trailing end is visible prior to insertion into the lateral flow test device 102. In other embodiments, the leading end of the assay test strip may not emerge through the lateral flow test device 102 and, instead, the exit may be blocked such that insertion and removal of the assay test strip 104 may be from the same single aperture on one side of the lateral flow test device 102.

FIGS. 2A and 2B show internal components in a detailed set-up 200 of the lateral flow test device 102 in accordance with the present disclosure, prior to insertion of the assay test strip 104. The lateral flow test device 102 comprises a test chamber 204 having a detection aperture 206. An optical detector 208 is configured to receive light from the assay test strip 104 through the detection aperture 206, when the assay test strip 104 is provided in the test chamber 204. The test chamber 204 is configured for manual feed of at least a portion of the assay test strip 104 passed the detection aperture 206 and the optical detector 208 is configured to detect one or more tracking features associated with the assay test strip 104 so as to determine when at least a test line 230 on the assay test strip 104 is detectable through the detection aperture 206.

The optical detector 208 is in the form of a spectral sensor mounted on a printed circuit board (PCB) 210, including control electronics, which in turn is housed in a lateral flow test device body 202. A user interface (not shown) may be connected to the PCB 210 to allow user input and control of the lateral flow test device 102.

The test chamber 204 is configured to prevent or at least minimise ambient light from reaching the detection aperture 206. This ensures that reliable and accurate results can be detected against a constant black backdrop. In particular, the test chamber 204 has an entrance aperture 216 for entrance of the assay test strip 104 into the device and an entrance obscurer 218 for preventing or at least minimising ambient light from entering the entrance aperture 216. The test chamber 204 also has an exit aperture 220 for exit of the assay test strip 104 from the device and an exit obscurer 222 for preventing or at least minimising ambient light from entering the exit aperture 220.

As shown in FIG. 2A, the entrance obscurer 218 and the exit obscurer 222 are in the form of a brush hair curtain. In other embodiments, the entrance obscurer 218 and/or the exit obscurer 222 may comprise a screen or another form of single or double curtain (e.g. of fabric or foam).

The test chamber 204 also has sidewalls (not shown), a chamber base 214a and a chamber top wall 214b forming a dark rectangular cuboid box with a lateral slit in the chamber top wall 214b forming the detection aperture 206.

As shown in FIG. 2B, a single optical emitter 212 in form of a white light source LED emitter is configured to emit light onto the assay test strip 104 when provided in the test chamber 204. Other forms of optical emitter 212 may be provided depending on the optical characteristics being detected. Similarly, other optical detectors 208 may be provided to detect a desired wavelength of light.

The optical emitter 212 is shown in FIG. 2B as being laterally spaced from the optical emitter 212 in an arrangement configured for reflectance of light from the optical emitter 212, to the optical detector via the assay test strip 104 (when inserted). In other embodiments, the optical emitter 212 may be configured for transmission of light from the optical emitter 212, to the optical detector via the assay test strip 104 (when inserted) or the optical emitter 212 may be configured for excitation of particles to enable detection of fluorescence (with use of appropriate filters). In some embodiments, such as those relying on luminescence of particles in the test line, no optical emitter may be required for detection of the particles of interest.

As illustrated in FIGS. 2A and 2B, the assay test strip 104 comprises the test line 230 and a control line 232 spaced along the assay test strip from the sample port 106 with the control line 232 begin provided furthest from the sample port 106. This ensures, that, as long as flow from a sample deposited in the sample port 106 reaches the control line 232, the flow must have passed through the test line 230. As explained above, the assay test strip will be provided with a conjugate intended to create a chemical reaction between the target molecule from the sample (e.g., an antigen or analyte) and its chemical partner (e.g. antibody or receptor). The sample and conjugate mix then flows along the assay test strip by capillary action, the analyte is bound on the bio-active particle and this complex is accumulated on the test strip 230 causing the test strip 230 to become visible or detectable by the optical detector 208. The control line 232 is configured to detect the conjugate (i.e. even if no analyte is present in the sample), thus providing an indicator that the flow has passed through the test line 230 even if the analyte is not detected. Typically, the control line 232, which is always detectable on valid test is detected first. In combination with a detected tracking speed of the assay test strip (e.g. determined from detection of the tracking features) the position of the test line 230 can be determined. Once the position is determined, an intensity of the received light from the test line 230 can be analysed. In another approach the detector reads (i.e. takes a series of measurements) across the full extent of the test strip 230 and analyses the received intensity information in accordance with the tracking speed information. The tracking speed information may observe 1) up and down movements of the assay test strip 104 within the device 102 and 2) variations in speed of the assay test strip 104 as it is inserted or moved under the detection aperture 206.

FIG. 3A shows a simplified side view 300 of the set-up of FIG. 2A, with the assay test strip 104 inserted such that the control line 232 is detectable through the detection aperture 206 in the chamber top wall 214b.

FIG. 3B shows a simplified side view 300 of the set-up of FIG. 2A, with the assay test strip 104 inserted such that the test line 230 is detectable through the detection aperture 206 in the chamber top wall 214b.

However, when the assay test strip 104 has been inserted into the enclosed dark test chamber 204 in the lateral flow test device 102, it is not possible for the user to visibly check that the control line 232 or test line 230 are correctly aligned for detection through the detection aperture 206. As such, the optical detector 208 is configured to detect one or more tracking features associated with the assay test strip 104 so as to determine when at least the test line 230 on the assay test strip 104 is detectable through the detection aperture 206. Further details of the tracking features are described in more detail below.

FIG. 4 shows a flow diagram of the steps involved in a method 400 of operating a lateral flow test device 102 in accordance with the present disclosure. The method 400 may comprise a set-up step (not shown) of switching the device 102 on and ensuring it is in a reading (data collection) mode. A first step 402 is then carried out of manually feeding at least a portion of an assay test strip 104 passed a detection aperture 206 in a test chamber 204 followed by a second step 404 of detecting, using an optical detector 208, one or more tracking features associated with the assay test strip 104. A third step 406 comprises determining, based on the detection of the one or more tracking features, when at least a test line 230 on the assay test strip 104 is detectable through the detection aperture 206. In a fourth step 408, the method comprises receiving light, at the optical detector 208, from at least the test line 230 of the assay test strip 104. In some embodiments, in a subsequent step (not shown), the received light (e.g. collected data) is analysed and a correlation is made between an intensity of the received light and the movement of the assay test trip 104 as derived from the tracking features. In some embodiments, steps 404 and 408 may occur concurrently or at substantially the same time (e.g. during a data collection phase when the assay test strip is swept through the device) and step 406 may occur subsequently (e.g. during a data analysis phase). In other words, the steps may be carried out in a different order to that illustrated.

An advantage of the method 400 is that the optical detector 208 can be used to determine the correct location of the test line 230 when the assay test strip 230 is enclosed in a dark test chamber 204 so that a reliable and accurate test result can be obtained. Furthermore, a single optical emitter 212 and a single optical detector 208 may be employed to detect light from any number of test/control lines whilst also enable tracking of the assay test strip 104 to ensure correct alignment for measurement of each test/control line. Moreover, the movement of the assay test strip 104 can be done manually without complex controls and equipment, thereby ensuring the lateral flow test device 102 remains cost-effective, especially for disposable tests.

Thus, the assay test strip 104 comprises one or more tracking features to aid location of the test line 230 (at least) such that the test line 230 is detectable through the detection aperture 206 of the lateral flow test device 102. The tracking features may comprise one or more of: a mark, a symbol, a shape, a protuberance, an indentation, a cutout, a colour, a material, a roughness, a thickness, a transparency or another property of the assay test strip 104 that is detectable by the optical detector 208.

For example, the assay test strip 104 may comprise one or more tracking features (e.g. marks) towards a leading edge or a side of the assay test strip 104, for example, laterally aligned with the test line 230 and control line 232. In which case, the optical detector 208 may be configured to detect the marks to ensure that the test line 230 and/or control line 232 is aligned with the detection aperture 206 either prior to taking a test measurement or when identifying a relevant measurement from a number of measurement samples.

In some embodiments, the lateral flow test device 102 may be configured to track the position of the assay test strip 104 using a technique similar to that employed in an optical computer mouse tracking system. Accordingly, the lateral flow test device 102 may be configured to use digital image correlation to track a position of the assay test strip 104 within the test chamber 204. This may involve using the optical detector 208 to take successive images of the assay test strip 104 as it is moved through the lateral flow test device 102. When light from the optical emitter 212 hits the surface of the assay test strip 104, at a grazing angle, distinct shadows are cast due to surface texture (e.g. roughness) of the assay test strip paper. The shadows are captured in the successive images, which are compared to determine how far the assay test strip paper has moved between images. In this way, it is possible to measure distance along the assay test strip 104 such that the location of the test line 230 and control line 232 can be accurately determined.

It should be noted that the lateral flow test device 102 may be configured to continually take images of the assay test strip 104 as it is inserted or passed through the device, with the PCB 210 electronics using digital image correlation to determine which of the images taken correspond to those of interest for taking a measurement from the test line 230, for example. In other words, the assay test strip 104 does not need to be specifically aligned with the detection aperture 206 for a measurement to be taken as an appropriate measurement can be extracted from the many images taken as the assay test strip 104 is swept through the device. The other images (i.e. those taken when the test line 230 is not aligned with the detection aperture 206) may be discarded.

The lateral flow test device 102 may be calibrated for a particular type (e.g. roughness) of assay test strip paper and may be pre-programmed with information regarding the position of the test line 230 and/or control line 232 on the assay test strip 104.

In some embodiments, the optical detector 208 may be configured to detect two or more spaced apart tracking features associated with the assay test strip 104 and the lateral flow test device 102 may be further configured to determine a speed of movement of the assay test strip 104 based on a time of detection of each of the two or more tracking features. The device 102 may detect variations in speed movement and may even determine a direction of the movement of the assay test strip 104 (e.g. backwards or forwards) through the device 102.

It will be understood that, in accordance with the disclosure, an assay test strip 104 with any number of lines (e.g. test lines 230 or control lines 232) can be read out from a single assay test strip 104 with a minimal set of components (e.g. one optical emitter 212 and one optical detector 208). By inserting or sweeping the assay test strip 104 through the lateral flow test device 102 the responses (e.g. fluorescence or reflectance) can be accurately read out. The speed of the sweeping movement can be determined by the optical detector 208, using a technique similar to that of an optical computer mouse tracking its position. Accordingly, if no signal is detected from a test line 230, it is possible to determine whether the test line 230 was measured properly (e.g. a reading was taken with the test line 230 in the correct location for detection through the detection aperture 206 for true nil result and not just misaligned). For example, this may be determined by detection of the control line 232, which in any case should become visible, in combination with the tracking speed information. In some embodiments, this can be determined by detection of a leading edge of the assay test strip 104 (on insertion) in combination with the tracking speed information. This latter case is especially useful if the control line 232 is not detectable.

Embodiments of the present disclosure can be employed in many different applications including biological applications, for example, in the areas of medical, environmental and veterinary diagnostics. For example, the lateral flow test device 102 may be configured as a point-of-care device. In some embodiments, the lateral flow test device 102 may be configured to detect coronavirus or coronavirus antibodies.

### LIST OF REFERENCE NUMERALS

100 schematic set-up
102 lateral flow test device
104 assay test strip
106 sample port
108 sample
200 detailed set-up
202 lateral flow test device body
204 test chamber
206 detection aperture
208 optical detector
210 PCB
212 optical emitter
214a chamber base
214b chamber top wall
216 entrance aperture
218 entrance obscurer
220 exit aperture
222 exit obscurer
230 test line
232 control line
300 simplified view
400 method of operation
402 step 1
404 step 2
406 step 3
408 step 4

The skilled person will understand that in the preceding description and appended claims, positional terms such as ‘above’, ‘along’, ‘side’, etc. are made with reference to conceptual illustrations, such as those shown in the appended drawings. These terms are used for ease of reference but are not intended to be of limiting nature. These terms are therefore to be understood as referring to an object when in an orientation as shown in the accompanying drawings.

Although the disclosure has been described in terms of preferred embodiments as set forth above, it should be understood that these embodiments are illustrative only and that the claims are not limited to those embodiments. Those skilled in the art will be able to make modifications and alternatives in view of the disclosure, which are contemplated as falling within the scope of the appended claims. Each feature disclosed or illustrated in the present specification may be incorporated in any embodiments, whether alone or in any appropriate combination with any other feature disclosed or illustrated herein.

1. A lateral flow test device comprising:
a test chamber having a detection aperture; and
an optical detector configured to receive light from an assay test strip through the detection aperture, when the assay test strip is provided in the test chamber;
wherein the test chamber is configured for manual feed of at least a portion of the assay test strip passed the detection aperture; and
wherein the optical detector is configured to detect one or more tracking features associated with the assay test strip so as to determine when at least a test line on the assay test strip is detectable through the detection aperture.

2. The lateral flow test device of claim 1, wherein the tracking features comprise one or more of: a mark, a symbol, a shape, a protuberance, an indentation, a cutout, a colour, a material, a roughness, a thickness, a transparency or another property of the assay test strip.

3. The lateral flow test device of claim 1, wherein the optical detector is further configured to determine when a control line on the assay test strip is detectable through the detection aperture.

4. The lateral flow test device of claim 1, wherein the test chamber is configured to prevent or minimize ambient light from reaching the detection aperture.

5. The lateral flow test device of claim 4, wherein the test chamber comprises an entrance aperture for entrance of the assay test strip into the device and an entrance obscurer for preventing or minimizing ambient light from entering the entrance aperture.

6. The lateral flow test device of claim 5, wherein the entrance obscurer comprises one or more of: a curtain, a screen and a brush.

7. The lateral flow test device of claim 4, wherein the test chamber comprises an exit aperture for exit of the assay test strip from the device and an exit obscurer for preventing or minimizing ambient light from entering the exit aperture.

8. The lateral flow test device of claim 7, wherein the exit obscurer comprises one or more of: a curtain, a screen and a brush.

9. The lateral flow test device of claim 1, further comprising a single optical emitter configured to emit light onto the assay test strip when provided in the test chamber.

10. The lateral flow test device of claim 9, configured to use digital image correlation to track a position of the assay test strip within the test chamber.

11. The lateral flow test device of claim 1, wherein the optical detector is configured to detect two or more tracking features associated with the assay test strip and the device is further configured to determine a speed of insertion of the assay test strip based on a time of detection of each of the two or more tracking features.

12. The lateral flow test device of claim 1, wherein a single optical detector is provided to detect the one or more tracking features and to receive light from at least the test line of the assay test strip.

13. The lateral flow test device of claim 1, configured as a point-of-care device.

14. The lateral flow test device of claim 1, configured to detect one or more of: coronavirus; coronavirus antibodies.

15. A method of operating a lateral flow test device comprising:
manually feeding at least a portion of an assay test strip passed a detection aperture in a test chamber;
detecting, using an optical detector, one or more tracking features associated with the assay test strip;
determining, based on the detection of the one or more tracking features, when at least a test line on the assay test strip is detectable through the detection aperture; and
receiving light, at the optical detector, from the test line of the assay test strip.

16. An assay test strip for use with the lateral flow test device of claim 1, wherein the assay test strip comprises at least a test line and one or more tracking features to aid location of the test line such that the test line is detectable through the detection aperture of the device.
